(19) United States
(12) Patent Application Publication
(10) Pub. No.: US 2009/0246519 A1
(43) Pub. Date: Oct. 1, 2009

(54) Biosynthesis of Metalloid Containing Nanoparticles by Aerobic Microbes

(75) Inventor: Thomas E. Hanson, Newark, DE (US)

Correspondence Address: RATNERPRESTIA, P.O. BOX 1596, WILMINGTON, DE, 19899, US

(73) Assignee: University of Delaware, Newark, DE (US)

(21) Appl. No.: 12/410,119

(22) Filed: Mar. 24, 2009

Related U.S. Application Data
(60) Provisional application No. 61/072,035, filed on Mar. 27, 2008.

(51) Int. Cl. C12P 3/00; C12N 1/20; C12N 1/16; B32B 5/16; C02F 3/00

(52) U.S. Cl. 428/364; 435/252.1; 435/255.1; 435/168; 428/402; 210/601; 977/762; 977/773; 977/810; 977/894

(57) ABSTRACT

Isolated tellurite-resistant or selenite-resistant marine organisms capable of precipitating tellurium or selenium when grown aerobically are described. A method for using these isolated organisms to produce an aqueous suspension of purified nanoparticles comprising tellurium or selenium and the nanoparticles comprising tellurium or selenium produced by this method are also described. The nanoparticles may further comprise cadmium or zinc. A method of remediation utilizing the described organisms is also presented.

### CROSS REFERENCE TO RELATED APPLICATIONS

This application claims priority to provisional application number U.S. 61/072,035, filed Mar. 27, 2008, which is incorporated herein, in entirety, by reference.

### STATEMENT REGARDING FEDERALLY SPONSORED RESEARCH

This invention was made with Government support under OCE-0425199 from the National Science Foundation. The Government has certain rights in this invention.

### BACKGROUND OF THE INVENTION

Highly pure tellurium (Te) and selenium (Se) are valuable to the electronic and semiconductor industries. Tellurium is an extremely rare metallic element that is a p-type semiconductor and also has fluorescence properties (e.g., CdTe quantum dots; (R. E. Bailey, et al., 2004, J Nanosci Nanotechnol 4: 569-574; S. K. Batabyal, et al., 2006, J Nanosci Nanotechnol 6: 719-725; N. I. Chalmers, et al., 2007, Appl Environ Microbiol 73: 630-636; T. J. Fountaine, et al., 2006, Mod Pathol 19: 1181-1191; K. P. Jayadevan and T. Y. Tseng, 2005, J Nanosci Nanotechnol 5: 1768-1784). Tellurium is used, for example, in microcircuitry, re-writable discs, memory chips, and thermoelectric devices. Currently there is a need for converting microcircuitry components to nanoscale circuitry components that require purified, nanoscale particles of tellurium. Selenium also has semiconductor properties, and is used in photovoltaic and photoconductive applications as well as in the manufacture of glass and ceramics, and as a chemical catalyst. Purified, nanoscale selenium particles are necessary for scaling down photovoltaics to nanoscale components. Tellurium an selenium each exhibit high fluorescent yield that does not fade upon excitation. Therefore, when alloyed with cadmium or zinc they are useful as quantum dot fluorophores, which are applied, for example, in biomedical imaging.

Microbial resistance to the inorganic oxyanion tellurite (TeO₃²⁻) is a widespread phenomenon. In most environments sampled to date, tellurite-resistant organisms comprise about 10% of the total culturable microbial population (C. N. Rathgeber, et al., 2002, Appl Environ Microbiol 68: 4613-4622; D. E. Taylor, 1999, Trends Microbiol 7: 111-115). Tellurite-resistant microbes have long been known to precipitate tellurium, but known tellurite-resistant organisms are strictly or facultatively anaerobic bacteria. The same is true for selenium precipitating bacteria. (D. S. Lee et al., 2007, Chemosphere 68:1898-1905, Yee et al., 2007, Appl Environ Microbiol 73:1914-1920, Astratinei et al., 2006, J Environ Qual. 35:1873-1883). However, the need for hypoxic or anoxic conditions to produce elemental tellurium or selenium hinders the use of these anaerobic organisms in large scale production of these materials.

### SUMMARY OF THE INVENTION

The invention provides an isolated tellurite-resistant and/or selenite-resistant marine organism capable of precipitating tellurium or selenium when grown aerobically. The isolated marine organism may be selected from the group of deposited organisms having ATCC accession numbers PTA-8965, PTA-8966, and PTA-8967.

Further provided is a method for producing nanoparticles comprising tellurium, selenium or a combination of tellurium and selenium comprising the steps of
a) culturing one or more of the tellurite- or selenite-resistant organisms under aerobic conditions in a medium containing soluble compounds comprising tellurium or selenium or a combination of tellurium and selenium;
b) culturing the organisms in the medium for a period sufficient for the organisms to precipitate nanoparticles comprising tellurium or selenium or a combination of tellurium and selenium;
c) extracting the nanoparticles from the organisms; and
d) recovering the extracted nanoparticles.

Also provided is a nanoparticle comprising tellurium, selenium or a combination of tellurium and selenium, produced by the provided method. The nanoparticle may also comprise cadmium or zinc. A method for removing compounds comprising tellurium, selenium, or arsenic from a liquid by adding the tellurite- or selenite-resistant organisms to the liquid is additionally provided.

### BRIEF DESCRIPTION OF THE DRAWINGS

FIG. 1. Resistance of model strains cultured in the absence of tellurite to varying concentrations of sodium tellurite in LB-marine plates. The viable population observed on LB-marine plates without tellurite was designated to be 100%. A) Cluster 1=strains 1A, 13B, 30B; B) Cluster 2=strains 6A, 28A; C) Cluster 3=strain 14B. Symbols for each strain are noted in the legend. Data points are the average of two independent experiments for each strain.

FIG. 2. Phylogenetic affiliations of isolates of tellurite-resistant strains based on 16s and 18s ribosomal DNA (rDNA). Isolates are indicated by their cluster and strain number in bold text. Individual sequences are noted by their GenBank identifier and strains isolated from marine environments are noted in italics. Major clades aside from those containing tellurite-resistant strains have been collapsed for clarity. The percentage of times each node was observed in 1000 bootstrapped replicates is noted and indicates that this tree and the taxonomic assignments derived from it are of high confidence. Scale bars indicate the numbers of substitutions per site. A) Combined 16S/18S rDNA tree including isolates from Cluster 1. B) Bacterial 16S rDNA sequence tree including isolates from Clusters 2 and 3.

FIG. 3. Recoveries of soluble and precipitated Te in model strains for each cluster. Te was determined by GF-AAS in the soluble and particulate fractions of each culture. A total of 0.65 mM Te was added to cultures of strains 13B (cluster 1, A) and 28A (cluster 2, B) while the culture of strain 14B received 0.16 mM Te (cluster 3, C). Dark bars denote tellurium recovered as precipitates; light bars represent tellurium recovered in the liquid. Each bar is the mean of four measurements per time point. The error bars represent the mean±standard deviation.

FIG. 4. Localization of precipitated Te in model strains from each cluster. Representative TEM images are displayed for model strains grown in the presence (+) or absence (−) of tellurite. Images are arranged in rows with the strain indicated at the left edge of each row and growth condition noted at the top of each column. A seven-fold magnified subsection (indicated by white box) of the +TeO₃²⁻ image for each strain is shown in the last column.

### DETAILED DESCRIPTION OF THE INVENTION

As described more thoroughly in Examples 1 and 2 below, obligately aerobic, highly tellurite-resistant microbes have been isolated for the first time from salt marsh sediments. The isolated strains segregate into three categories based on colony morphology and degree of tellurite resistance as shown in Table 1. Phylogenetic analysis demonstrates that these strains are either eukaryotes of the genus Rhodotorula or prokaryotes of the Bacillales, closely related to marine Bacillus spp. and distinct from B. selenitireducens (E. A. Gontang, et al., 2007, Appl Environ Microbiol 73: 3272-3282). All strains examined efficiently precipitated high concentrations of pure tellurium (Te) or selenium (Se) under aerobic conditions. These isolated microbe strains are further described in P. L. Oliver, et al., 2008, Appl Env Microbiol 74: 7163-7173, which is incorporated herein, in entirety, by reference. Elemental Te precipitates were the dominant end product of tellurite metabolism and accumulated intracellularly. Although it was expected from the literature that a range of Gram-negative organisms would dominate these isolations, in fact, all the isolated strains stained Gram-positive.

Three strains of the isolated, tellurite-resistant marine microbes were deposited with the American Type Culture Collection (ATCC) on Feb. 21, 2008 by Dr. Thomas E. Hanson on behalf of the University of Delaware, and assigned the following ATCC designations on Mar. 4, 2008:

PTA-8965 Virgibacillus halodenitrificans: 14B

PTA-8966 Bacillus sp.: 6A

PTA-8967 Rhodoturula mucilaginosa: 1A.

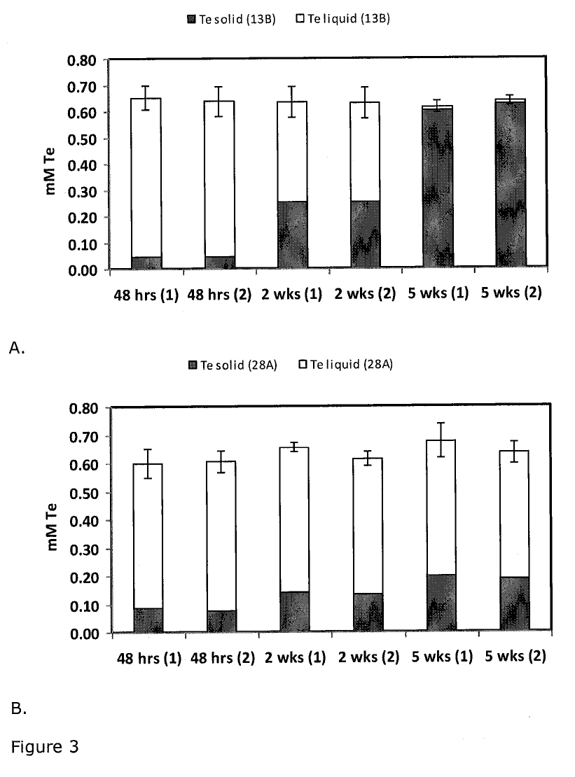

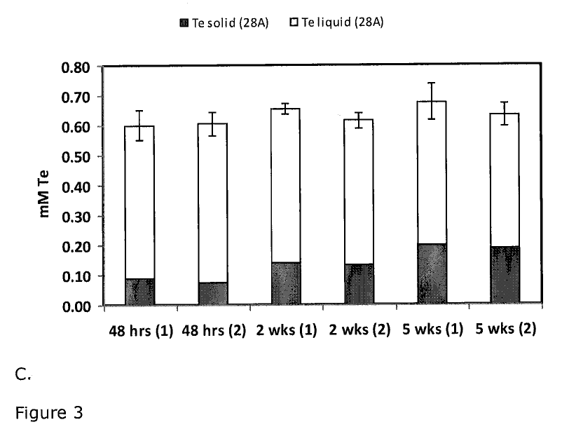

The isolates described here precipitate greater quantities of tellurium than representative Gram negative bacteria reported in the literature. For example, Basnayake et al. observed 34% conversion of 0.1 mM tellurite to solid Te by Pseudomonas fluorescens K27 (R. S. T. Basnayake, et al., 2002, Appl Organomet Chem 15: 499-510). In comparison, over five weeks, cluster 1 strains converted about 95% of 0.7 mM tellurite, while cluster 2 strains converted about 40% of 0.7 mM tellurite and cluster 3 strains converted about 10-15% of 0.2 mM tellurite (FIG. 3). These results show that the marine Bacillus spp. is generally more effective at tellurite conversion to particulate Te than Gram negative bacteria.

To allow the isolated microorganisms (also referred to herein as “cells”) to produce pure tellurium or pure selenium nanoparticles, the organisms are grown aerobically in an appropriate medium to which doses of sodium tellurite or sodium selenite are added. One embodiment of the culture method is discussed in detail in Example 6. In general, cultures are incubated at room temperature or the culture temperature may be optimized for a particular strain of organism. The cultures are generally agitated to maintain aerobic conditions. The organisms are cultured in medium without tellurite or selenite for a period of time sufficient to yield an optimal number of organisms for the efficient production and isolation of nanoparticles. As known in the art, this time period will depend, in part, on the size of the culture, the rate of growth of the organism, and the amount of inoculum.

Sodium tellurite or sodium selenite may be added to the medium as a single dose or, preferably, in multiple, smaller doses. Toxicity to the microorganisms is reduced and more rapid accumulation of particulate Te in the cells engendered by adding lower concentrations/dose of tellurite or selenite to the cultures in multiple doses (about 65% of 0.1 mM tellurite precipitated in 48 hours). The final applied total of tellurite may range from 0.05 mM to 68 mM (11 mg/L to 15,000 mg/L), which is the solubility limit for tellurite. The final applied total concentration of selenite may range from 0.05 mM to 100 mM (9 mg/L to 17,294 mg/L).

In one embodiment of the invention, organisms were harvested about 24 h after the last dose of Te or Se by centrifugation. The organisms may be harvested by any appropriate means that separates them from the culture medium, e.g., centrifugation, filtration.

The harvested organisms are then treated in a manner that allows extraction of the nanoparticles from the cells. This may include osmotic cell lysis by resuspension in a hypotonic buffer, physical cell lysis by grinding, sonication, or pressure, and/or chemical cell lysis utilizing solutions of detergents (i.e. sodium dodecylsulfate) and/or cell wall degrading enzymes (i.e. lysozyme) at ambient or elevated temperatures. Nanoparticles are then separated from the cell debris and isolated by any appropriate method, such as centrifugation or filtration. Generally it is desirable to wash the isolated nanoparticles in an appropriate solvent, e.g., water, ethanol, buffer. The purified nanoparticles may be quantified by any appropriate method, e.g., mass, elemental analysis, or mass spectrometry, and stored as a liquid suspension. The suspension should remain wetted, as once particles are dried, they become tightly adherent and are difficult to disperse. The particle suspension can be stored at room temperature or frozen under ambient atmospheric conditions.

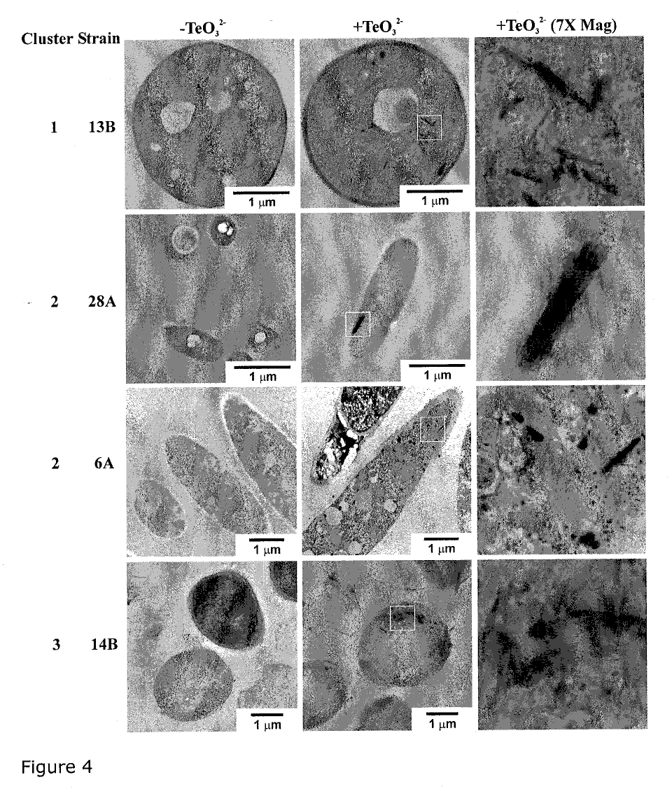

In one embodiment, the isolated nanoparticles ranged in size from less than 10 nm to greater than 50 nm in diameter for nanospheres and up to 300 nm in length for needles or wires, based on electron micrographs such as those shown in FIG. 4. Different strains produce different shapes of tellurite nanoparticle precipitates (FIG. 4), suggesting that crystal growth properties can be tailored to a given application. For example, needle-like particles are suitable for use as wires and spherical particles are suitable as connectors in nanoscale circuitry.

In other embodiments, purified nanoparticles of tellurium and/or selenium combined with cadmium or zinc, which may be produced, for example, as described in Example 8, can be used as quantum dot fluorophores in biomedical imaging and other applications. The quantum dot compounds CdSe, CdTe, and ZnTe are also semiconductors.

Elemental Te and Se are p-type semiconductors and display piezoelectricity, the ability to produce electric current when deformed (C. Métraux and B. Grobéty, 2004, J Mater Res 19: 2159-2164). Piezoelectric materials have diverse applications in acoustics, atomic force microscopy, and ignition devices, for example, cigarette lighters.

Semiconducting nanoparticles are essential components of thermoelectric materials that incorporate organic materials (P. Reddy et al., 2007, Science 315: 1568-1571; www.lbl.gov/tt/techs/Ibnl2380). Therefore, the materials described here may find wide application in miniaturized electronics, solar cells, and piezoelectric devices. Elemental Te nanostructures are also used as seed materials for the synthesis of platinum-rich and platinum-rich carbonaceous nanostructured materials that have potential uses in electrochemistry, fuel cells, sensors, and other fields. (B. Zhang et al., 2007, Adv Funct Mater 17: 486-492).

An additional application of the disclosed organisms that follows naturally from the precipitation of Te and Se is their use in the remediation of selenium, tellurium, or arsenic contaminated waters or waste streams (Example 9). The conversion of the environmentally mobile and toxic forms of Te/Se to relatively non-toxic and immobile elemental Te/Se by microbes has been proposed (Astratinei et al., 2006, J Environ Qual 35: 1873-1883, and references therein). Bacterial remediation of selenium contamination is currently being tested for feasibility in agricultural wastewaters (Y. Zhang, et al., 2008. Biores Technol. 99: 1267-1273, and references therein). However, the organisms described herein provide particular advantages for remediation in terms of their ability to tolerate high metalloid concentrations that are more likely to be present in industrial waste streams, as well as the “bonus” of providing a source of purified nanoparticles of Te, Se or combinations of these elements. For example, these organisms can convert toxic Te/Se compounds at ambient temperatures and pressures. No specialized equipment, supervision, or control mechanisms are required. In addition, because these organisms grow aerobically, no special measures need be employed to exclude oxygen from the remediation system.

### EXAMPLES

### 1. Isolation and Growth of Strains

An optimized medium, LB-marine, contained per liter: 2.0 g tryptone, 1.0 g yeast extract, 12.5 g sodium chloride, and 1 mL of trace element solution (described in T. M. Wahlund, et al., 1991, Arch. Microbiol. 156: 81-90). This mixture was adjusted to pH 8.1, 1.5% (w/v) agar added for plates when desired, and autoclaved for 15 minutes at 121° C. After cooling, 20 ml of sterile 1M magnesium sulfate was added per liter of medium prior to pouring plates or inoculating liquid cultures. Tellurite was added to the medium from concentrated filter-sterilized stocks after autoclaving and was employed at 150 μg ml⁻¹ to isolate resistant strains.

Mud samples, from the upper 2 cm of sediment, were collected from fringing salt marsh bordering the Indian River inlet, in Rehoboth Beach, Del. in May of 2004. Mud was suspended 1:10 (v/v) in 0.45 μm filter sterilized water collected at the sampling site and transported to the laboratory. Enrichments were incubated under aerobic conditions at room temperature. Some enrichments were amended with tellurite supplied as 150 μg Na₂TeO₃ ml⁻¹.

Strains were isolated from primary dilutions of the mud enrichments on LB-marine agar plates at room temperature both in the presence and absence of 150 μg Na₂TeO₃ ml⁻¹. Single tellurite-resistant colonies were purified by restreaking until a single colony morphology was consistently obtained. Purified strains were grown in liquid medium at 30° C. with shaking at 250 rpm, and frozen glycerol stocks prepared for long term storage at −70° C. Strains were revived from glycerol stocks by streaking onto LB-marine plates with tellurite.

### 2. Tellurite Resistance Determination

Tellurite resistance of strains was assessed by culturing strains in liquid LB-marine medium in the absence of tellurium. Cell concentrations in liquid cultures were determined by direct counting using a Hausser counting chamber (Fisher Scientific, Pittsburgh, Pa.). Cultures were diluted to about 2×10³ cells ml⁻¹ and 100 μl plated on LB-marine plates without amendment or containing variable concentrations of Na₂TeO₃ ranging from 75 to 1200 μg ml⁻¹. Plates were incubated for at least two weeks to allow for the observation of slow growing colonies. Colonies were usually observed on plates within five days.

Gram stains of culture or colony smears were performed with commercial reagents (Protocol Gram stain, VWR, West Chester, Pa.) according to manufacturer's instructions. Stained samples were observed on an Olympus (Central Valley, Pa.) BX61 microscope equipped with a UApo/340 40× objective.

The total culturable population of aerobic microbes recovered on LB-marine in the absence of tellurite selection averaged 1.2×10⁴ colony forming units (CFU) ml⁻¹ in 1:10 sediment slurries indicating a culturable population of 1.2×10⁵ CFU per ml of original sediment. The total number of tellurite-resistant organisms recovered was 9.0×10³ CFU ml⁻¹ in sediment slurries, indicating an initial population size of 9.0×10⁴ CFU ml⁻¹ tellurite-resistant strains in the original sediment. Thus, about 8% of the total culturable population was found to be tellurite-resistant. Enrichment with tellurite in sediment slurries for periods of up to two weeks increased the proportion of tellurite-resistant strains by two-fold, to about 15% of the total culturable microbial population (data not shown).

When isolated strains from LB-marine without tellurite were patched onto plates containing 150 μg Na₂TeO₃ ml⁻¹, 8% of these strains were found to be tellurite-resistant, duplicating the original fraction of tellurite resistance observed in the initial isolation experiment. All tellurite-resistant strains from the original isolation grew in the absence of tellurite and maintained their tellurite resistance. A total of 30 strains were colony purified by repeated streaking on LB-marine plus tellurite and carried forward for characterization.

### 3. Characterization of Tellurite Resistant Strains

Tellurite-resistant isolates were grouped initially on the basis of colony morphology and subsequently characterized for their tellurite resistance range on LB-marine plates (Table 1). Based on these two criteria, the thirty isolates could be divided into three clusters. Six representative model strains from these clusters were carried forward to further examine their properties (Table 1).

TABLE 1
Clustering of tellurite-resistant isolates based on isolate properties.

| | | Cell | Maximum | | Model |
|---|---|---|---|---|---|
| Cluster | Colony Morphology | Morphology | [TeO₃²⁻]^{a} | | Strains |
| 1 | −TeO₃²⁻ compact, smooth, rose pink | Ovoid | 600 | μg ml⁻¹ | 1A, 13B, |
| | +TeO₃²⁻ compact, black, minute at | | | | 30B |
| | 600 μg ml⁻¹ | | | | |
| 2 | −TeO₃²⁻ large, undefined edge, pale | Rod | 300 | μg ml⁻¹ | 28A, 6A |
| | orange | | | | |
| | +TeO₃²⁻ large, black in center, grey | | | | |
| | on edge, | | | | |
| | minute at 150 μg ml⁻¹ | | | | |
| 3 | −TeO₃²⁻ compact or spreading, buff | Rod | 75-150 | μg ml⁻¹ | 14B |
| | or white | | | | |
| | +TeO₃²⁻ minute, grey | | | | |

^{a}Values are the highest levels of tellurite tolerated by strains when grown on LB-Marine plates

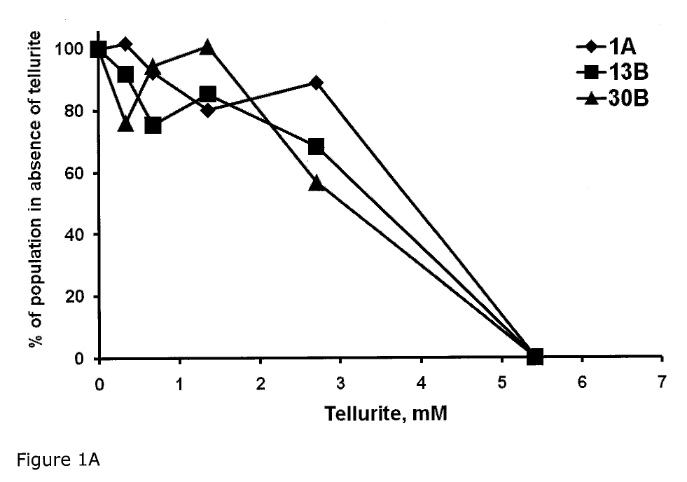

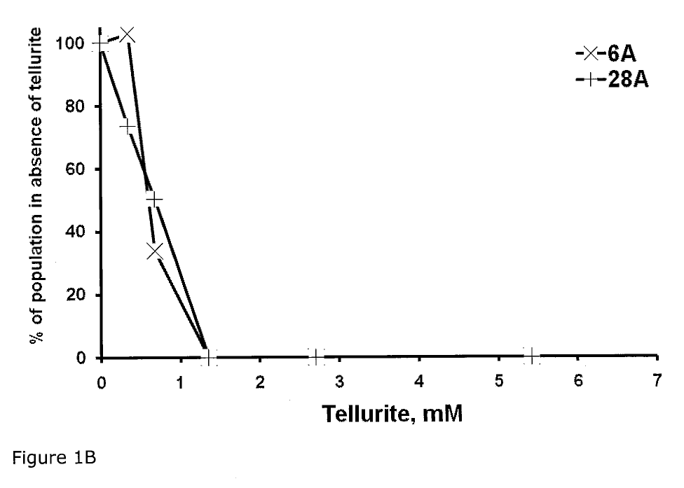

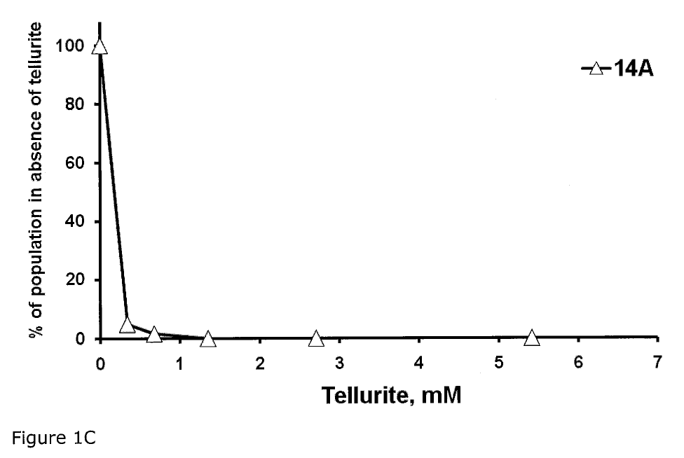

Cluster 1 is composed of highly tellurite-resistant isolates (FIG. 1A) that form compact, non-spreading rose pink colonies. On plates containing tellurite, these colonies are dark black in color. Cluster 2 is composed of isolates that display moderate tellurite resistance (FIG. 1B). These organisms have variable colony morphology. One of the model strains for this cluster, strain 6A, forms moderately sized, white colonies with a fungal appearance. In contrast, strain 28A forms large, shiny, pale orange colonies in the absence of tellurite and grey to black minute colonies in the presence of tellurite. Cluster 3 is composed of isolates that display relatively weak tellurite resistance (FIG. 1C). Even though strain 14B, the cluster 3 model strain, was isolated in the presence of 150 μg Na₂TeO₃ ml⁻¹, it grows poorly at this concentration both on plates and in liquid cultures. Therefore, this strain was routinely propagated in the presence of 37.5 μg Na₂TeO₃ ml⁻¹. Colonies in the absence of tellurite are buff colored or white. In the presence of tellurite, colony size is greatly diminished and colonies were colored slightly gray. Liquid cultures of this strain tend to grow as gelatinous aggregates, rather than the dispersed cultures typical of the other isolates.

All tellurite-resistant strains isolated to date in this study stained Gram positive. Isolated colonies recovered on LB-marine in the absence of tellurite selection contained both Gram positive and Gram negative organisms with nearly equal frequencies (data not shown). Thus, it appears that a specific subset of Gram positive organisms was identified by tellurite selection and that the Gram negative organisms in the upper sediment layers sampled were not tellurite-resistant under the conditions tested. Strains in all clusters were also resistant to 0.7 mM tellurate, selenate, selenite, arsenate, and arsenite, (equivalent to 150 μg Na₂TeO₃ ml⁻¹) under aerobic growth conditions (data not shown).

All strains described here were isolated as aerobes and are able to grow under tellurite selection at full atmospheric oxygen tension, which distinguishes them from B. selenitireducens and B. aresnicoselenatis.

### 4. Phylogenetic Assignment of Isolates

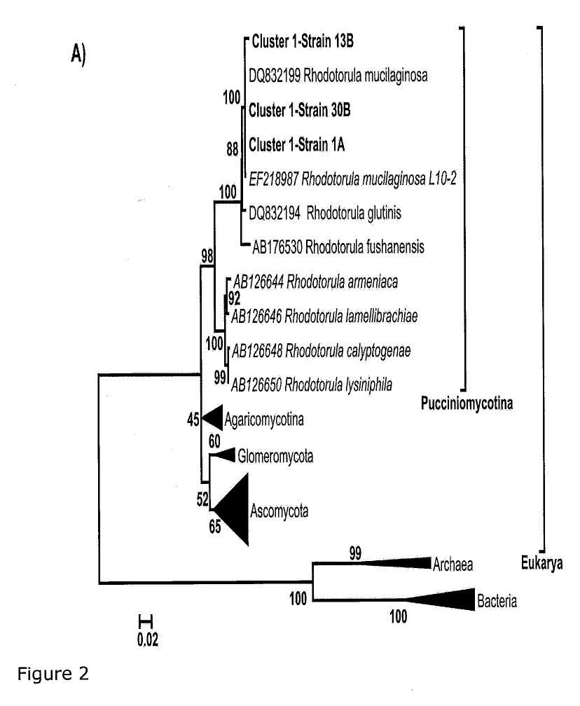

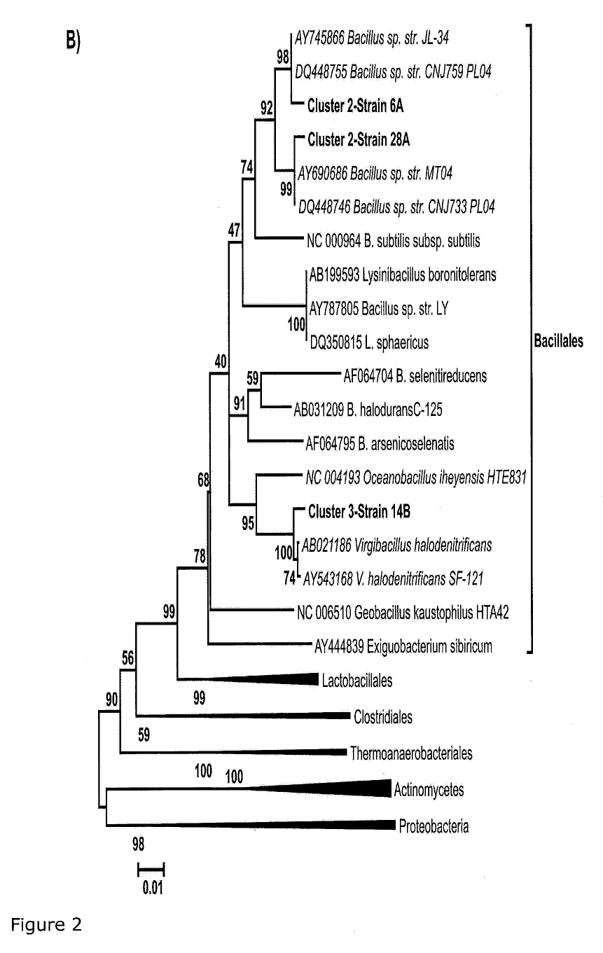

An approximately 900 base pair fragment of ribosomal DNA (rDNA) was PCR-amplified from each of the six model strains in Table 1, then cloned, and sequenced according to standard methods. Phylogenetic relationships among the isolates were determined as described in P. L. Ollivier, et al., 2008, Appl Env Microbiol 74: 7163-7173 and are shown in FIG. 2. Comparison of cluster 1 rDNA sequences to those in known databases indicated that these strains are all eukaryotes related to the yeast genus Rhodotorula mucilaginosa, strains of which are frequently isolated from marine and estuarine sediments. Comparison of rDNA sequences from the isolates within clusters 2 and 3 unambiguously identified them as members of the family Bacillaceae, order Bacillales of the class Bacilli within the phylum Firmicutes of Gram positive bacteria. Cluster 2 strains were most closely related to various uncharacterized marine Bacillus isolates (E. A. Gontang, et al., 2007, Appl Environ Microbiol 73: 3272-3282). The cluster 3 strain 14B was most closely related to strains of Bacillus halodenitrificans (syn. Virgibacillus halodenitrificans (J. H. Yoon, et al., 2004, Int. J. Syst. Evol. Microbiol 54: 2163-2167) and Oceanobacillus iheyensis (J. Lu, et al., 2001, FEMS Microbiol. Lett. 205: 291-297). None of the strains closely related to those identified here has previously been reported as resistant to tellurium, selenium or arsenic oxyanions in the literature. The isolates produced by this study very likely represent new sub-species or strains of recognized organisms as they display ≧99.5% nucleotide sequence identity with their closest counterparts in sequence databases.

### 5. Quantification of Solid and Dissolved Tellurium Species

Tellurium content in liquid and solid phases of samples was determined using a Perkin-Elmer (Waltham, Mass.) Model 3300 Atomic Absorption Spectrometer equipped with a graphite furnace accessory HGA-600 (GF-AAS) and an autosampler. A hollow cathode lamp was employed as emission source at 214.3 nm with a slit width of 2 nm and 30 mA lamp current. Measurements were performed in peak area (integrated absorbance) mode. Tubes with pyrolytic graphite coating were used throughout the experiments. High purity argon was used as the internal gas. The temperature-time program was performed according to M. Y. Shiue, et al., 2001, J Analyt Atomic Spectr 16: 1172-1179). The formation of tellurium oxides TeO (g) during pyrolysis can lead to analyte losses (G. M. Muller-Vogt, 2000, Spectrochimica Acta Part B-Atomic Spectroscopy 55: 501-508). To overcome this issue, a 20 μl aliquot of the sample (i.e. 0.5-2 ng Te) was injected into the furnace followed by 20 μl of palladium (30 μg ml⁻¹, i.e. 0.6 μg Pd) mixed with magnesium (200 μg ml⁻¹, i.e. 4 μg Mg) matrix modifier. With these techniques, a linear range was found between 0-100 ppb Te (0-2 μg ml⁻¹) using a commercially prepared standard solution (Aldrich Chemical, Milwaukee, Wis.) in 5% (v/v) HNO₃.

Well-mixed culture samples were centrifuged (9,000×g, 25 min) and the supernatant transferred to a teflon beaker where it was evaporated to dryness. The residue was dissolved with suboiled HNO₃, dried again, and redissolved in 5% (v/v) HNO₃. The pellet was resuspended with Te-free media and collected again by centrifugation. When this wash supernatant was analyzed as above, the tellurium was less than 1% of that measured in the original supernatant. Pellets were dissolved with suboiled HNO₃ (decolorization and dissolution was immediate), dried in Teflon beakers, and dissolved in 5% (v/v) HNO₃. Samples from the supernatant or pellet were diluted by factors ranging from 5- to 210-fold in order to obtain tellurium concentrations between 25 and 100 ppb. Samples containing no added tellurium were analyzed with each batch of samples to estimate the level of potential contamination introduced by lab operations. In all cases these were indistinguishable from the background level.

Total recovery of Te in the soluble and particulate fractions ranged from 80-110% of the amended Te in any given measurement, with a mean of 95±6%. Cluster 1 strains appear to be more efficient at precipitating Te, converting ˜98% of added Te to a particulate form (FIG. 4A) while cluster 2 and 3 strains only converted 30-40% of added Te to a particulate form (FIGS. 4B&C) over five weeks of culture.

### 6. Process for Nanoparticle Production

To demonstrate the feasibility of scaling this process up from the small volumes used in prior experiments, two liter cultures of tellurite-resistant strains were prepared in LB-marine medium in two-liter vacuum flasks sealed with rubber stoppers having two sections of tubing that penetrated the stopper. One piece of tubing was used to deliver filter-sterilized, humidified air and the other piece of tubing was used to remove samples from the flasks to monitor growth and to add doses of sodium tellurite or sodium selenite. Air and volatile products exited the culture via the vacuum arm of the flask. Cultures were incubated in a fume hood at room temperature and mixed by stirring with a stir bar at 350 rpm.

Cultures were inoculated with a 1% (v/v) inoculum from a dense pure culture of each noted strain and grown for 48 hours in the absence of tellurite or selenite. Thereafter, cultures were dosed every 24 hours with solutions of sodium tellurite or sodium selenite. One day after the final dose of tellurite or selenite, cells were harvested from the cultures by centrifugation.

To isolate pure Te and Se nanoparticles from the cells, cell pellets were resuspended in a 2% (w/v) solution of sodium dodecylsulfate in water and heated at 100-105° C. to lyse the cells. Nanoparticles were recovered by centrifugation and washed extensively with water before quantifying their recovery by mass after drying overnight at 75° C. Yields of Te and Se nanoparticles are shown in Table 2.

TABLE 2
Recovery of Te and Se nanoparticles from two liter scale up cultures.

| | | | | Total | | |
|---|---|---|---|---|---|---|
| | Com- | Te/Se | No. | Te/Se | Te/Se | % |
| Strain | pound | per Dose | Doses | Applied | Recovered | Recovery |
| 13B | Na₂TeO₃ | 171 mg | 4 | 684 mg | 216 mg | 31.5 |
| 6A | Na₂SeO₃ | 108 mg | 4 | 433 mg | 315 mg | 72.7 |

### 7. Localization of Precipitated Tellurium

Culture samples were observed by phase contrast microscopy on an Olympus (Central Valley, Pa.) BX61 microscope equipped with a UPlan Fl 40× Ph2 objective and phase ring set. Images were acquired with a RETIGA EXi CCD camera (QImaging, Surrey, B. C., Canada) and stored as TIFF files.

Cells from cultures were harvested by centrifugation and fixed with 2% glutaraldehyde and 2% paraformaldehyde in 0.1M Na cacodylate (primary fixative) and 1% OsO₄ (secondary fixative). Resin infiltration was carried out with Embed-812 (Electron Microscopy Sciences, Hatfield, Pa.). Blocks were sectioned on a Reichert-Jung Ultra-cut E Microtome (Leica Microsystems, Bannockburn, Ill.) with a diamond knife. Thin sections were approximately 60-70 nm (silver interference color) and were collected on copper grids (Electron Microscopy Sciences, Hatfield, Pa.). The sections were post-stained with uranyl acetate and methanol as well as Reynolds' lead citrate (E. S. Reynolds, 1963, J Cell Biol 17: 208). The samples were viewed using a Zeiss (Goettingen, Germany) CEM 902 transmission electron microscope at 80 kV, and images taken with a Soft Imaging System Mega View II (Olympus Soft Imaging, Lakewood, Colo.).

As particulate Te is the dominant product of tellurite metabolism in all strains examined, we sought to determine where the Te precipitate was localized. Cultures were examined directly by phase contrast microscopy to determine if they were producing extracellular crystalline materials, but no significant amounts were observed in any of the strains (data not shown). Thin sections of fixed cells were examined by TEM and strains in all clusters found to contain electron dense bodies that were only present when strains were cultured in the presence of tellurite (FIG. 5). Generally, these electron dense bodies were found evenly distributed throughout the cell sections. This lack of distinct localization indicates that the Te is precipitated intracellularly without any obvious membrane association. Strain 28A in cluster 2 was the exception to this rule as it tended to form precipitates in regions close to the cell periphery, suggesting a membrane localization for the tellurium precipitation activity in this strain.

Isolated strains appeared to produce different shapes and sizes of precipitates. Cluster 1 strains generally formed clusters of short needles <100 nm in length, though individual cells sometimes contained clusters over 300 nm in length. X-ray diffraction characterization of elemental Te particles produced by strain 13B further indicate that the material is crystalline. The images of strain 13B support the eukaryotic affiliation of cluster 1 strains inferred from 18S rDNA sequencing, as nuclei and mitochondria were clearly distinguishable in most sections. Strains in cluster 2 displayed more variability in the Te precipitate structure. Strain 6A formed spheres and amorphous aggregates that ranged from the <10 to >50 nm in diameter. In contrast, strain 28A precipitates were primarily observed as aggregates of needles at the cell periphery a few hundred nm in length (strain 28A). Precipitates produced by the cluster 3 strain 14B were less electron dense and less compact than those of other strains. This may be due to the relatively low tellurite (37.5 μg Na₂TeO₃ ml⁻¹, 0.18 mM) levels required for the growth of strain 14B in liquid culture and the tendency of this strain to aggregate in culture. Aggregation may either protect cells by exclusion of tellurite leading to lower intracellular concentrations for precipitation.

### 8. Preparation of CdTe and CdSe

Synthesis of quantum dot fluorophores CdTe and CdSe would be accomplished by the simultaneous application of solutions of CdCl₂ and Na₂TeO₃ or Na₂SeO₃ to cultures as described for the synthesis of elemental Te and Se above. In addition, both Na₂TeO₃ and Na₂SeO₃ can be simultaneously applied to cultures and both elements precipitated at the same time. Dosing of these compounds into cultures would be optimized for each particular combination desired by evaluating microbial growth in the presence of multiple substrates using standard microbial growth experiments.

### 9. Remediation of a Contaminated Liquid

The organisms described above can be grown on site in a dedicated flow-through bioreactor where contaminated waters are continually added to the reactor containing appropriate growth medium and the organism. The hydraulic retention time of the reactor would be adjusted until the metalloid concentration in the reactor effluent meets regulatory targets. The population size of the organisms for this application would be >1×10⁶ cells/ml. This is commonly known as a “pump and treat” application. In addition, as these organisms are natural isolates, they could be employed in cultures for bioaugmentation. Bioaugmentation is the process of adding a microbial culture directly to a contaminated system along with appropriate carbon sources. In the case of these aerobic, tellurite- and selenite-resistant organisms, any complex carbon source similar in content to yeast extract and tryptone could be employed. Bioaugmentation is particularly effective in ground water systems where the organisms can be added via injection wells. A typical inoculum for bioaugmentation would be 10-200 L of culture at a density of about 1×10⁸⁻⁹ cells/ml prior to injection into the site.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. An isolated tellurite-resistant or selenite-resistant marine organism capable of precipitating tellurium or selenium when grown aerobically.

2. The isolated marine organism of claim 1, wherein the organism is a bacterium or a yeast.

3. The isolated marine organism of claim 2, wherein the organism is selected from the genera consisting of Virgibacillus, Bacillus, and Rhodotorula.

4. The isolated marine organism of claim 3, wherein the organism is selected from the group of deposited organisms having ATCC accession numbers PTA-8965, PTA-8966, and PTA-8967.

5. A method for producing nanoparticles of tellurium, selenium or a combination of tellurium and selenium comprising the steps of
a) culturing one or more of the isolated tellurite- or selenite-resistant marine organisms of claim 1 under aerobic conditions in a medium containing soluble compounds comprising tellurium, selenium or a combination of tellurium and selenium;
b) incubating the organisms in the medium for a period sufficient for the organisms to precipitate nanoparticles comprising tellurium, selenium, or a combination of tellurium and selenium;
c) extracting the nanoparticles from the organisms; and
d) recovering the extracted nanoparticles.

6. A nanoparticle comprising tellurium, selenium or a combination of tellerium and selenium produced by the method of claim 5.

7. The nanoparticle of claim 6, wherein the nanoparticle comprises a nanowire or a nanosphere.

8. The nanoparticle of claim 6, wherein the nanoparticle comprises tellurium.

9. The nanoparticle of claim 6, wherein the nanoparticle comprises selenium.

10. The method of claim 5, wherein the medium further comprises compounds comprising cadmium or zinc.

11. A nanoparticle comprising tellurium, selenium, or a combination of tellurium and selenium, further comprising cadmium or zinc, produced by the method of claim 10.

12. A method for removing a compound comprising one or more elements selected from the group consisting of tellurium, selenium and arsenic from a liquid comprising combining the isolated tellurite-resistant or selenite-resistant marine organism of claim 1 with the liquid.

13. The method of claim 12, wherein the compound is selected from the group consisting of tellurite, tellurate, selenite, selenate, arsenite, and arsenate.

14. The method of claim 12 further comprising adding a carbon source appropriate for the isolated tellurite-resistant or selenite-resistant marine organism to the liquid.

15. The method of claim 12, wherein the isolated tellurite-resistant or selenite-resistant marine organism is maintained in a bioreactor and the liquid flows through the bioreactor.

16. The method of claim 12, wherein the liquid is a natural body of water or an industrial waste stream.
